(19) United States
(12) Patent Application Publication
(10) Pub. No.: US 2026/0174913 A1
(43) Pub. Date: Jun. 25, 2026

(54) NOVEL METHOD FOR PREPARING TESTOSTERONE DERIVATIVES BY SOLID-PHASE SYNTHESIS

(71) Applicant: KING FAISAL SPECIALIST HOSPITAL & RESEARCH CENTRE, RIYADH (SA)

(72) Inventor: Subhani Mohammad OKARVI, RIYADH (SA)

(21) Appl. No.: 18/999,860

(22) Filed: Dec. 23, 2024

(51) Int. Cl. A61K 51/04; C07B 59/00; C07J 1/00

(52) U.S. Cl. CPC A61K 51/0493; A61K 51/0497; C07B 59/007; C07J 1/0029; A61K 2121/00; A61K 2123/00; C07B 2200/05

(57) ABSTRACT

The present invention relates to a method of preparing a novel acetic acid testosterone. The present invention further relates to a novel acetic acid testosterone, preferably obtained by the method of the present invention. The present invention further relates to a method of preparing a testosterone derivative and/or a testosterone derivative for labeling, preferably prepared from said acetic acid testosterone. The present invention further relates to the testosterone derivative and/or a testosterone derivative for labeling, preferably obtained by the method of the present invention, as well as to the use of the testosterone derivative and/or a testosterone derivative for labeling, for radiolabeling with diagnostic and/or therapeutic radionuclides and the medical uses.

### FIELD OF THE INVENTION

The present invention relates a novel acetic acid testosterone and to a kind of new DOTA-testosterone or NODAGA-testosterone or AoA-testosterone preparation method, that belongs to radiopharmaceutical chemistry art.

### BACKGROUND OF THE INVENTION

Steroid hormone receptors are important molecular targets for anticancer therapies, particularly for patients with breast and prostate cancer. One such receptor, the androgen receptor (AR) is emerging as an important factor in the pathogenesis of breast cancer, which is the most common malignancy among females worldwide. Recent research indicates that the AR receptor is a potential clinical and emerging hormonal target in breast cancer, with potential clinical benefit in both estrogen receptor (ER) positive and negative tumors. Compared to the ER, AR contains unique functional domains with relevance to its altered role in human breast cancer. The majority of ER-positive tumors express AR, and a significant percentage of ER-negative tumors might benefit from combined targeting of AR and the ErbB2/HER2 oncogene. The accordance of more than 70% of AR expression in primary and metastatic breast tumors implies that AR may be a new marker and a potential therapeutic target among AR-positive breast cancer patients.

The actions of androgens, such as testosterone and dihydrotestosterone are mediated via the androgen receptor, a ligand-dependent nuclear transcription factor and member of the steroid hormone nuclear receptor family. Androgens are expressed at different levels in men and women, and while they are important for proper development, they can also drive tumor growth. Most notably, the role of AR in prostate cancer has been extensively studied. Recent data highlight the effectiveness of AR as an emerging hormonal target in breast cancer. Breast cancer is characterized by heterogeneity at the molecular and clinical levels. Due to the complexity of breast cancer, it is no longer considered a single disease, and the need for sub-classification has emerged. The main breast cancer subtypes have been classified according to their molecular profile and several established biomarkers, including estrogen receptor, progesterone receptor, and human epidermal growth receptor-2. While estrogens play the biggest role in female breast development, androgens are also crucial in this process. Interestingly, it has been noticed that the androgen receptor is expressed in 70 to 90% of breast cancer incidents and that it plays a vital role in breast cancer pathology and progression. In triple-negative breast cancer (TNBC), AR positivity may represent more than 50% of cases, and its expression levels vary considerably among TNBC molecular subtypes. Although AR is implicated in all stages of breast cancer development, its function seems to vary among different breast cancer subtypes. AR expression might also play a role during tumor progression to metastatic disease. Androgen receptor has been proposed as a potential therapeutic target for breast cancer, and the availability of AR inhibitors approved for prostate cancer treatment could constitute a therapeutic tool for specific subsets of breast cancer.

Testosterone is a male sex hormone that binds to ARs, which are overexpressed in prostate and breast carcinoma. In males, testosterone is produced in the testes whereas in females it is synthesized in the ovaries and adrenal glands. In female breast tissue, testosterone is converted into dihydrotestosterone (DHT) or 17β-estradiol, and binds onto AR or ERα, leading to the inhibition or stimulation of cell proliferation, respectively. Radiolabeled compounds specifically designed to image breast and prostate tumors based on the androgen content (i.e., testosterone) may serve as a useful clinical tool in staging the disease and monitoring the course of hormonal therapy. The development of several potent receptor binding agents combined with sophisticated molecular imaging techniques has greatly advanced this field, including single photon emission computed tomography (SPECT) and positron emission tomography (PET). With the advent of modern molecular imaging, it is now possible to detect breast cancer in its early stages, determine the extent of the disease, administer appropriate therapeutic protocols, and monitor the effects of treatment. Many tumors of the breast and prostate contain receptors for androgens, and the presence and levels of the receptors can usually be correlated with the endocrine responsiveness of the cancer. These receptors also provide a potential mechanism whereby suitably radiolabeled androgens could be selectively concentrated in a manner that would permit imaging of the tumor, thus providing an assessment of the stage of the cancer and its potential for endocrine responsiveness, diagnostic and prognostic information of importance in the selection of the most favorable treatment regimen. These receptors should be particularly useful targets for imaging in patients who are on endocrine therapy. Visualization of AR in the tumor using an AR-based imaging agent might provide a better representation of the AR status of the tumor. By precisely characterizing the tumor properties and biological processes involved, state-of-the-art modern molecular imaging can play an important role in minimizing the morbidity and mortality associated with breast cancer. Progress in the field of functional imaging will possibly lead to more specific tumor targeting and personalized treatment, increasing tumor control and improving quality of life.

The initial developments of radiolabeled testosterone ligands involved the preparation of ¹¹C-methyltestosterone. In the last two decades, several ^{99m}Tc-labeled testosterone-based compounds were prepared and evaluated. The first ^{99m}Tc-labeled testosterone was developed by introducing the N₂S₂ (diamine-dithiol) donor atom set to coordinate the ^{99m}Tc. Additionally, various derivatives of androgens have been radiolabeled with several gamma-emitting radionuclides, such as ⁷⁷Br, ⁸²Br, ¹²⁵I, and ⁷⁵Se for receptor-based prostatic imaging agents. However, rapid metabolic cleavage, low receptor binding affinity, and inadequate specific activity are the major impediments to the use of these radiolabeled conjugates. Also, rhenium-containing testosterone derivative was developed by the synthesis of 17β-hydroxy-7α-(5-mercaptopent-1-yl)-androst-4-en-3-one and its conversion into the corresponding oxorhenium (V) complexes. Several ¹⁸F-labeled androgen compounds have been developed to image hormone receptors and include 16α-[¹⁸F]-fluoroestradiol ([¹⁸F]FES) for estrogen receptors, 21-[¹⁸F]fluoro-16α-ethyl-norprogesterone ([¹⁸F]FENP) for progesterone receptors and 16β-[¹⁸F]fluoro-5α-dihydrotestosterone ([¹⁸F]FDHT) for androgen receptors. Of these, [¹⁸F]FES has been most extensively studied and recently approved by the US FDA for imaging of ER-positive breast carcinoma. The development of testosterone analogs suitable for imaging breast cancer is lacking and this emerging field needs to be explored.

The synthesis of testosterone has been described in several publications. For example, DAHPES-testosterone conjugate was synthesized starting with the reaction of testosterone with aminooxy acetic acid to produce testosterone-3-(O-carboxymethyl) oxime followed by coupling of this intermediate with 5-hydroxy-3,7-diazanonan-1,9-dithiol (DAHPES) to yield DAHPES-testosterone conjugate. U.S. Pat. No. 2,609,378A describes the preparation of enolthioethers of testosterone and the process for the production of these ethers, the acyl esters for the production of testosterone, and acyl esters of testosterone. WO 2017/093980 A1 describes the process for the preparation of testosterone by preparing 4-androsten-3,17-dione that can be converted to testosterone. Other studies mentioned the preparation of 7α-functionalized testosterone derivatives. The key step in the synthesis involves the copper-catalyzed, α-selective 1,6-Michael addition of a 4-pentenylmagnesium bromide to 6-dehydrotestosterone 17β-acetate.

However, the available methods of preparing testosterone derivatives involve a large number of steps for the synthesis and/or the purification of a testosterone derivative. Thus, there is a need for a method of preparing a testosterone derivative in good purity that requires fewer synthetic steps with few purification steps. Also, there is a need for a method of preparing a testosterone derivative that does not require expensive reagents or large amounts of reagents and that involves a rapid synthesis of testosterone derivatives with short reaction times while maintaining good purity. This method should provide a flexible synthesis approach to enable the production of a testosterone derivative on a small scale (milligrams) or large scale (grams), making the method suitable for industrialized production of testosterone derivatives. There is also a need for a method of preparing a testosterone derivative with good solubility in an aqueous medium that can be conjugated to different bifunctional ligands or a testosterone derivative that can be radiolabeled with different radionuclides. Also, there is a need for new testosterone derivatives that can be used as a new theranostic agent for both imaging and therapeutic applications for androgen receptors expressing primary and metastatic cancers, such as breast cancer and other cancers. Furthermore, there is a need for precursor of testosterone derivatives, which can be used for the effective production of testosterone derivatives.

### SUMMARY OF THE INVENTION

One objective of the present invention was to provide a precursor of testosterone derivatives, which can be used for the effective production of testosterone derivatives. Also, an objective of the present invention was to provide a method of preparing testosterone derivative(s) in good purity that requires fewer synthetic steps with few purification steps. An objective of the present invention was to provide a method of preparing testosterone derivative(s) that does not require expensive reagents or large amounts of reagents and that involves a rapid synthesis of testosterone derivatives with short reaction times while maintaining good purity. This method should provide a flexible synthesis approach to enable the production of a testosterone derivative on a small scale (milligrams) or large scale (grams), making the method suitable for industrialized production of testosterone derivatives. An objective of the present invention was to provide a method of preparing a testosterone derivative with good solubility in an aqueous medium that can be conjugated to different bifunctional ligands or a testosterone derivative that can be radiolabeled with different radionuclides. An objective of the present invention was to provide new testosterone derivatives that can be used as a new theranostic agent for both imaging and therapeutic applications for androgen receptors expressing primary and metastatic cancers, such as breast cancer and other cancers.

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

One aspect the present invention is an acetic acid testosterone of the general formula 1

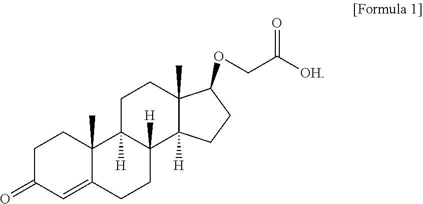

In one embodiment the acetic acid testosterone is prepared by reacting testosterone and bromo acetic acid; wherein, preferably, testosterone and bromo acetic acid are reacted in the presence of potassium carbonate (K₂CO₃) and potassium iodide (KI).

In one embodiment, the acetic acid testosterone is prepared by reacting testosterone and bromo acetic acid, wherein the reaction is first carried out at a temperature in a range from about 60° C. to about 100° C., preferably from about 80° C. to about 90° C. for a duration in a range from about 1 h to about 10 h, preferably about 2 h to about 6 h, most preferably for about 4 h,
and then the reaction is carried out at a temperature in a range from about 18° C. to about 30° C., more preferably from about 20° C. to about 25° C., even more preferably at ambient temperature, such as at about 23° C., for a duration in a range from about 12 h to about 24 h, preferably from about 16 h to about 20 h, most preferably for about 18 h.

In one embodiment, the acetic acid testosterone is used for a preparation of a testosterone derivative and/or a testosterone derivative for radiolabeling.

Another aspect of the present invention is a testosterone derivative and/or a testosterone derivative for radiolabeling, preferably prepared from the acetic acid testosterone according to the present invention, wherein the testosterone derivative and/or the testosterone derivative for radiolabeling has the general formula 2 or the general formula 3

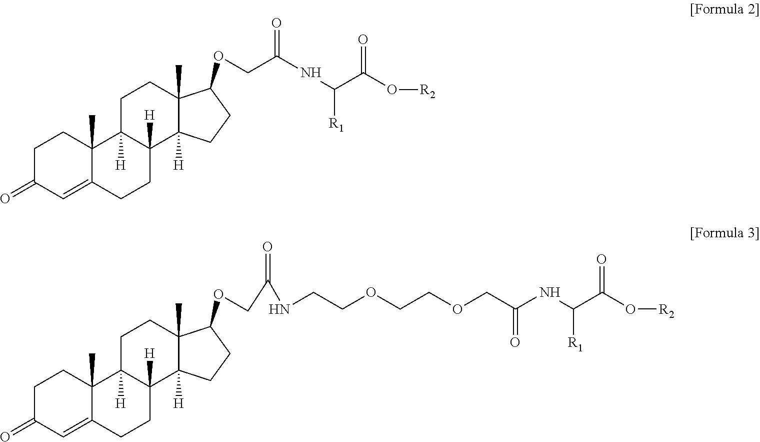

wherein R₁ is selected from a group comprising a lysine side chain, a modified lysine side chain, and a bifunctional group for radiolabeling coupled to a lysine side chain;
wherein, preferably, the modified lysine side chain is a lysine side chain having a protective group, such as ivDde (1-(4,4-dimethyl-2,6-dioxocyclohex-1-ylidene)-3-methylbutyl), or Alloc (Allyloxycarbonyl); more preferably ivDde;
   wherein, further preferably, the bifunctional group for radiolabeling is coupled to the amino group of the lysine side chain;
   wherein R₂ is selected from a group comprising a resin for solid-phase synthesis, preferably Wang resin, and H.

In one embodiment, the bifunctional group for radiolabeling is selected from DOTA (1,4,7,10-tetraazacyclododecane-1,4,7,10-tetraacetic acid), NODAGA (1,4,7-triazacyclononane-1-glutaric acid-4,7-diacetic acid), and AoA (aminooxyacetic acid).

In a preferred embodiment, the testosterone derivative and/or the testosterone derivative for radiolabeling has the general formula 4 or the general formula 5

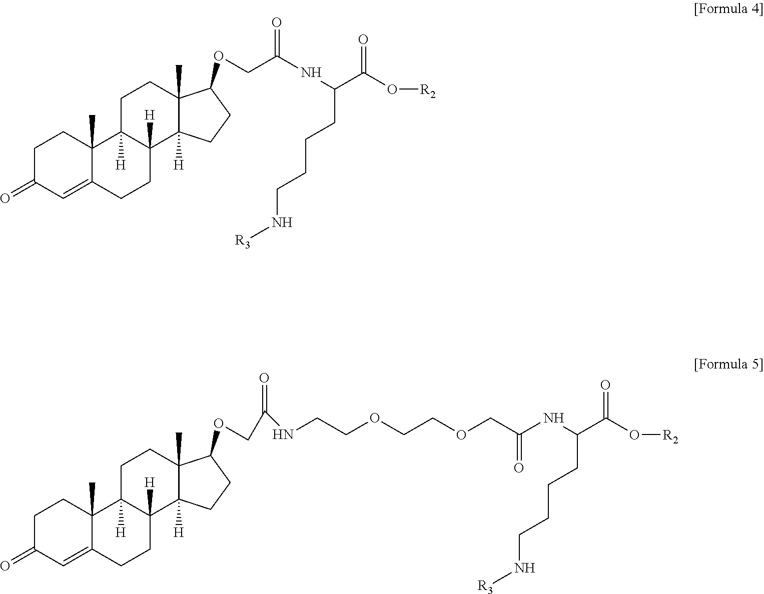

wherein R₃ is selected from a group comprising H, a protective group, and a bifunctional group for radiolabeling;
wherein, preferably, the protective group is ivDde (1-(4,4-dimethyl-2,6-dioxocyclohex-1-ylidene)-3-methylbutyl), or Alloc (Allyloxycarbonyl); more preferably ivDde;
wherein, further preferably, the bifunctional group for radiolabeling is selected from the group comprising DOTA (1,4,7,10-tetraazacyclododecane-1,4,7,10-tetraacetic acid), NODAGA (1,4,7-triazacyclononane-1-glutaric acid-4,7-diacetic acid), and AoA (aminooxyacetic acid);
wherein R₂ is selected from a group comprising a resin for solid-phase synthesis, preferably Wang resin, and H.

In one embodiment, the testosterone derivative and/or the testosterone derivative for radiolabeling is radiolabeled with diagnostic and/or therapeutic radionuclides(s), such as ⁶⁸Ga, ¹⁷⁷Lu, ⁶⁴Cu, ⁸⁹Zr, or ¹⁸F, and/or diagnostic radiotracer(s), such as ¹⁸FDG (fluorodeoxyglucose).

In one embodiment, wherein the testosterone derivative and/or the testosterone derivative for radiolabeling is selected from
DOTA-testosterone,
NODAGA-testosterone,
AoA-testosterone,
DOTA-PEG-testosterone
NODAGA-PEG-testosterone, and
AoA-PEG-testosterone;
wherein, preferably, the testosterone derivative and/or the testosterone derivative for radiolabeling is selected from
DOTA-testosterone,
NODAGA-testosterone,
AoA-testosterone, and
DOTA-PEG-testosterone;
wherein, optionally, the testosterone derivative further comprises diagnostic and/or therapeutic radionuclides(s), such as ⁶⁸Ga, ¹⁷⁷Lu, ⁶⁴Cu, ⁸⁹Zr, or ¹⁸F, and/or diagnostic radiotracer(s), such as ¹⁸FDG (fluorodeoxyglucose).

Another aspect of the present invention is a method of preparing a testosterone derivative and/or a testosterone derivative for radiolabeling, wherein the method comprises the steps of
(1) preparing an acetic acid testosterone according to the present invention;
(2) either
   (a) coupling the acetic acid testosterone obtained in step (1) to a solid-phase synthesis resin, preferably Wang resin, wherein said resin, preferably Wang resin, is suitable for Fmoc (9-fluorenylmethyl-oxycarbonyl)-solid-phase synthesis and carries a Fmoc-lysine with a protecting group at the lysine side chain;
      or
   (a′) first coupling Fmoc-NH-PEG-COOH (9 atoms; 4 equivalent) to a solid-phase synthesis resin, preferably Wang resin, wherein said resin, preferably Wang resin, is suitable for Fmoc (9-fluorenylmethyl-oxycarbonyl)-solid-phase synthesis and carries Fmoc-lysine with a protecting group at the lysine side chain to obtain a Fmoc-NH-PEG-resin, preferably a Fmoc-NH-PEG-Wang resin, and second, coupling the acetic acid testosterone obtained in step (1) to the Fmoc-NH-PEG-resin, preferably Fmoc-NH-PEG-Wang resin;
(3) removing the protective group at the lysine side chain;
(4) coupling a bifunctional group for radiolabeling to the lysine side chain; and
(5) removing the testosterone derivative and/or the testosterone derivative for radiolabeling from the resin.

In one embodiment, the protective group at the lysine side chain of the Fmoc-lysine is ivDde (1-(4,4-dimethyl-2,6-dioxocyclohex-1-ylidene)-3-methylbutyl) and the resin is Fmoc-Lys(ivDde)-Wang resin, or wherein the protective group at the lysine side chain of the Fmoc-lysine is Alloc (Allyloxycarbonyl) and the resin is Fmoc-Lys(Alloc)-Wang resin; wherein, preferably, the resin is Fmoc-Lys(ivDde)-Wang resin.

In one embodiment, prior to the coupling of step (2), the Fmoc group of the Fmoc-Lys(ivDde)-Wang resin is removed, preferably by treating the Fmoc-Lys(ivDde)-Wang resin with a piperidine solution;
wherein prior to the coupling of step (2) the acetic acid testosterone and/or the Fmoc-NH-PEG-COOH is preactivated at its α-carboxylic function by the use of an activating reagent; wherein, preferably, said activating reagent is HATU (hexafluorophosphate azabenzotriazole tetramethyl uronium) and oxyma pure (ethyl cyanohydroxyiminoacetate) in the presence of a base DIEA (diisopropylethylamine);
wherein additionally, in case of step (2) (a′), the Fmoc group of the Fmoc-NH-PEG-resin is removed prior to coupling of the preactivated acetic acid-testosterone to said Fmoc-NH-PEG-resin, preferably by treating the Fmoc-NH-PEG-resin with a piperidine solution.

In one embodiment, the bifunctional group for radiolabeling is selected from DOTA (1,4,7,10-tetraazacyclododecane-1,4,7,10-tetraacetic acid), NODAGA (1,4,7-triazacyclononane-1-glutaric acid-4,7-diacetic acid), and AoA (aminooxyacetic acid).

In one embodiment, in step (4), for coupling the bifunctional group for radiolabeling to the lysine side chain DOTA-tris-(t-Bu ester), NODAGA-tris-(t-Bu ester), or Boc-AoA is used,
wherein, preferably, said DOTA-tris-(t-Bu ester), NODAGA-tris-(t-Bu ester), or Boc-AoA is preactivated with an activating reagent, preferably with HATU in the presence of DIEA.

In one embodiment, at least one of steps (2) to (5) is carried out at a temperature in a range from about 18° C. to about 30° C., preferably from about 20° C. to about 25° C., more preferably at ambient temperature, such as at about 23° C.;
wherein, optionally, all steps are carried out at a temperature in a range from about 18° C. to about 30° C., preferably from about 20° C. to about 25° C., more preferably at ambient temperature, such as at about 23° C.

In one embodiment, the method further comprises a step of
(6) radiolabeling the testosterone derivative and/or the testosterone derivative for radio labeling obtained in step (5) with diagnostic and/or therapeutic radionuclide(s), such as ⁶⁸Ga, ¹⁷⁷Lu, ⁶⁴Cu, ⁸⁹Zr, or ¹⁸F,
   and/or diagnostic radiotracer(s), such as ¹⁸FDG (fluorodeoxyglucose).

In one embodiment of the present invention, the testosterone derivative and/or the testosterone derivative for radiolabeling according to the present invention is obtained by a method according to the present invention.

Another aspect of the present invention is a composition, preferably a pharmaceutical composition, comprising the testosterone derivative and/or the testosterone derivative, for radiolabeling according to the present invention.

Another aspect of the present invention is a use of the testosterone derivative and/or the testosterone derivative for radiolabeling according to the present invention, or the testosterone derivative and/or the testosterone derivative for radiolabeling prepared by the method according to the present invention, or the composition according to the present invention, for radiolabeling with diagnostic and/or therapeutic radionuclide(s), such as ⁶⁸Ga, ¹⁷⁷Lu, ⁶⁴Cu, ⁸⁹Zr, or ¹⁸F, and/or diagnostic radiotracer(s), such as ¹⁸FDG (fluorodeoxyglucose).

Another aspect of the present invention is the testosterone derivative and/or the testosterone derivative for radiolabeling according to the present invention, or the testosterone derivative and/or the testosterone derivative for radiolabeling prepared by the method according to the present invention, or the composition according to the present invention, for use in medicine.

In one embodiment, the present invention discloses the testosterone derivative and/or the testosterone derivative for radiolabeling according to the present invention, or the testosterone derivative and/or the testosterone derivative for radiolabeling prepared by the method according to the present invention, or the composition according to the present invention, for use in a method of imaging, diagnosis, and/or treatment of cancer; wherein, preferably, the cancer is an androgen-receptor-positive cancer; even more preferably an androgen-receptor-positive breast cancer or prostate cancer; most preferably an androgen-receptor-positive breast carcinoma or prostate carcinoma. Such use typically comprises administering the testosterone derivative and/or the testosterone derivative for radiolabeling to a patient in need thereof.

Another aspect of the present invention relates to a method of imaging, diagnosis and/or treatment of cancer in a patient with a compound derived from an acetic acid testosterone according to the present invention.

Another aspect of the present invention relates to a method of imaging, diagnosis and/or treatment of cancer in a patient with the testosterone derivative and/or the testosterone derivative for radiolabeling according to the present invention.

A further aspect of the present invention is the use of an acetic testosterone of the general formula 1 for the manufacture of a medicament for the imaging, diagnosis and/or treatment of cancer, preferably an androgen-receptor-positive cancer; even more preferably an androgen-receptor-positive breast cancer or prostate cancer; most preferably an androgen-receptor-positive breast carcinoma or prostate carcinoma.

A further aspect of the present invention is the use of the testosterone derivative(s) and/or the testosterone derivative(s) for radiolabeling in accordance with the present invention for the manufacture of a medicament for the imaging, diagnosis and/or treatment of cancer, preferably an androgen-receptor-positive cancer; even more preferably an androgen-receptor-positive breast cancer or prostate cancer; most preferably an androgen-receptor-positive breast carcinoma or prostate carcinoma.

### DETAILED DESCRIPTION

In one embodiment, the term “patient” relates to a human or an animal, preferably a human.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an Indefinite or definite article is used when referring to a singular noun, e.g. “a”, “an” or “the”, this includes a plural of that noun unless something else is specifically stated.

Furthermore, the terms “first”, “second”, “third” or “(a)”, “(b)”, “(c)”, “(d)” etc, and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The term “acetic acid testosterone”, as used herein refers to a molecule of the general formula 1 [Formula 1]

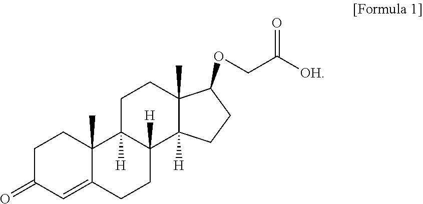

Preferably, acetic acid testosterone is prepared by reacting testosterone and bromo acetic acid; wherein, even more preferably, testosterone and bromo acetic acid are reacted in the presence of K₂CO₃ and potassium iodide (KI). The acetic acid testosterone, as described herein, may serve as a precursor for the production of (a) testosterone derivative(s) or (a) testosterone derivative(s) for radiolabeling as described herein.

The term “derivative”, as used herein, refers to a compound that is formed from a similar compound or a compound that can be imagined to arise from another compound, if one or more atoms are replaced with another atom or group of atoms. The term also encompasses structural analogs of a compound that can differ in one or more atoms, functional groups, or substructures, which are replaced with other atoms, groups, or substructures. A derivative or structural analog can be imagined to be formed, at least theoretically, from the other compound. Accordingly, the term “testosterone derivative”, as used herein, refers to a compound that is similar to testosterone, can be formed from testosterone, or a compound that can be imagined to arise from testosterone if one atom is replaced with another atom or group of atoms.

The term “radiolabeling”, as used herein, refers to a form of isotopic labeling that uses radionuclides or radio tracers. For the present application, radiolabeling should be understood to also encompass radiotracing. Radiolabeling can be used to track the movement of an isotope, e.g. in a microscopic cell or throughout chemical reactions. The term “radionuclide”, as used herein, refers to a nuclide that has excess numbers of either neutrons or protons, giving it excess nuclear energy, and making it unstable. A radionuclide can also be described as “radioactive nuclide”, “radioisotope”, or “radioactive isotope”. A radionuclide is an isotope of artificial or natural origin that shows radioactivity. The term “radiotracer”, as used herein, refers to a synthetic derivative of a natural compound in which one or more atoms have been replaced by a radionuclide. Radiotracers can also be referred to a “radioactive tracers” or “radioactive labels”. Radionuclides and/or radiotracers can serve as agents in nuclear medicine, play a role in computer imaging for diagnosis and experiment and may be used to destroy cells in tumor therapy. Radionuclides and/or radiotracers can further be used to track the distribution of a substance within a natural system such as a cell or tissue, as a flow tracer to track fluid flow, or to the localization of specific cell types and tissues. Examples for diagnostic radionuclides are ⁶⁸Ga, ¹⁷⁷Lu, ⁶⁴Cu, ⁸⁹Zr and ¹⁸F. An example for a diagnostic radiotracer is ¹⁸FDG (fluorodeoxyglucose).

The term “solid-phase synthesis”, as used herein, refers to a method in which molecules are covalently bound on a solid support material, a “solid-phase synthesis resin”, and can undergo assembly in a stepwise manner. This allows the reaction by-products to be removed at each step by simple washing. Examples for suitable “solid-phase synthesis resins” are Wang resins, such as Fmoc-Lys(ivDde)-Wang resin and Fmoc-Lys(Alloc)-Wang resin.

The term “Fmoc-solid-phase synthesis”, as used herein, is a form of solid-phase synthesis that uses a resin, preferably Wang-resin, that carries a Fmoc (9-fluorenylmethyl-oxycarbonyl)-lysine with a protecting group, at the lysine side chain. An example for a protecting group is ivDde (1-(4,4-dimethyl-2,6-dioxocyclohex-1-ylidene)-3-methylbutyl). During different synthesis steps, Fmoc and the other protecting groups may be removed to allow the reaction of the previously protected groups with other agents.

The term “protective group”, as used herein, refers to a temporary group introduced into a molecule by chemical modification of a functional group to prevent said functional group from reacting. Thereby, a protective group, sometimes also referred to as “protecting group”, helps to obtain chemoselectivity in a subsequent chemical reaction.

The term “activating agent”, as used herein, refers to a reagent used to prepare or excite a molecule and/or a functional group for a subsequent reaction.

The term “α-carboxylic function”, as used herein, refers to the ability of the carboxyl group of carboxylic acids to undergo chemical reactions with other molecules.

The term “androgen-receptor-positive”, or short “AR-positive”, as used herein, refers to the respective hormone receptor status. The hormone receptor status is considered positive if at least 15 to 20% of the tumor cells are receptor-positive. In the present application, the term “androgen receptor” (AR) may also be referred to as “testosterone receptor”. Unless stated otherwise, these terms may be used interchangeably. Likewise, the term “androgen-receptor-positive” may also be referred to as “testosterone-receptor-positive”.

The term “androgens”, as used herein, refers to hormones, such as testosterone, that are members of the steroid hormone receptor family of the nuclear receptor superfamily. The biological action of androgens is mediated through the androgen receptor (AR). Cancer cells that are androgen receptor-positive may need androgens to grow. These cells, also called AR-positive cells, may stop growing or die when they are treated with substances that block the binding and actions of androgen hormones.

The term “Wang resin”, as used herein, can also be referred to as “4-Benzyloxybenzyl alcohol polystyrene, polymer-bound”, “p-Alkoxybenzyl alcohol, polymer-bound”, or “[4-(Hydroxymethyl)phenoxymethyl]polystyrene”.

The term “Fmoc-Lys(Alloc)-Wang resin”, as used herein, can also be referred to as “Fmoc-Lys(Aloc)-Wang resin”, “Fmoc-L-Lys(Alloc)-Wang resin”, “Fmoc-L-Lys(Aloc)-Wang resin”, “Fmoc-L-Lys(Allyloxycarbonyl)-Wang resin”, “Fmoc-Lys(Allyloxycarbonyl)-Wang resin”, “Fmoc-Lys(Allyloxycarbonyl)-Wang resin”, “Fmoc-L-lysine (Alloc)-Wang resin”, or “Fmoc-lysine (Aloc)-Wang resin”.

The term “Fmoc-Lys(ivDde)-Wang resin”, as used herein, can also be referred to as “Fmoc-L-Lys(ivDde)-Wang resin” “Fmoc-L-lysine (ivDde)-Wang resin”, “Fmoc-lysine (ivDde)-Wang resin”, “Fmoc-Lys(1-(4,4-dimethyl-2,6-dioxocyclohex-1-ylidene)-3-methylbutyl)-Wang resin”, “Fmoc-L-Lys(1-(4,4-dimethyl-2,6-dioxocyclohex-1-ylidene)-3-methylbutyl)-Wang resin”, “Fmoc-L-lysine (1-(4,4-dimethyl-2,6-dioxocyclohex-1-ylidene)-3-methylbutyl)-Wang resin”, or “Fmoc-lysine (ivDde)-Wang resin”.

The term “oxyma pure”, as used herein, refers to a compound of the chemical formula CH₆N₂O₃. Oxyma pure can also be referred to as “ethyl cyanohydroxyiminoacetate” or “ethyl cyano(16yridine16ino)acetate”.

The term “HATU” (hexafluorophosphate azabenzotriazole tetramethyl uronium), as used herein, refers to a reagent used in peptide coupling chemistry to generate an active ester from a carboxylic acid. HATU may also be referred to as “1-[Bis(dimethylamino)methylene]-1H-1,2,3-triazolo[4,5-b]pyridinium 3-oxide hexafluorophosphate” or “N-[(Dimethylamino)-1H-1,2,3-triazolo-[4,5-b]16yridine-1-ylmethylene]-N-methylmethanaminium hexafluorophosphate N-oxide”. Preferably, HATU is used along with DIEA.

The term “DIEA” (diisopropylethylamine), as used herein, refers to an organic compound that is a tertiary amine. Preferably, DIEA is used in organic chemistry as a non-nucleophilic base. DIEA may also be referred to as “DIPEA”, “N,N-Diisopropylethylamine”, “Hünig's base”, “N-ethyl-N-(propan-2-yl)propan-2-amine”, “ethyldi(propan-2-yl)amine”, “N-ethyl-N-isopropylpropan-2-amine”, or “ethyldi(isopropyl)amine”.

The term “theranostics”, as used herein, refers to a combination of molecularly targeted imaging and therapy in which imaging provides actionable information that enables new or more effective therapies. Theranostics is the therapy-accompanying diagnosis with the aim of patient-specific therapy. One aim of theranostics is to provide the right therapy for the right patient at the right time.

Testosterone Derivatives and/or Testosterone Derivatives for Radiolabeling and their Uses

As outlined above, the present invention provides a testosterone derivative and/or a testosterone derivative for radiolabeling selected from:
DOTA-testosterone,
NODAGA-testosterone,
AoA-testosterone,
DOTA-PEG-testosterone
NODAGA-PEG-testosterone, and
AoA-PEG-testosterone.

Preferably the testosterone derivative and/or the testosterone derivative for radiolabeling is selected from:
DOTA-testosterone,
NODAGA-testosterone,
AoA-testosterone, and
DOTA-PEG-testosterone.

The term “DOTA-testosterone”, as used herein, may also be referred to as “DOTA-lysine-testosterone” or “Testosterone-lysine-DOTA”.

The term “NODAGA-testosterone”, as used herein, may also be referred to as “NODAGA-lysine-testosterone” or “Testosterone-lysine-NODAGA”.

The term “AoA-testosterone”, as used herein, may also be referred to as “AoA-lysine-testosterone” or “Testosterone-lysine-AoA”.

The term “DOTA-PEG-testosterone”, as used herein, may also be referred to as “DOTA-PEG-lysine-testosterone”, “Testosterone-lysine-DOTA-PEG”, or “Testosterone-PEG-lysine-DOTA”.

The term “NODAGA-PEG-testosterone”, as used herein, may also be referred to as “NODAGA-PEG-lysine-testosterone”, “Testosterone-lysine-NODAGA-PEG”, or “Testosterone-PEG-lysine-NODAGA”.

The term “AoA-PEG-testosterone”, as used herein, may also be referred to as “AoA-PEG-lysine-testosterone”, “Testosterone-lysine-AoA-PEG”, or “Testosterone-PEG-lysine-AoA”.

In a preferred embodiment, the testosterone derivative and/or the testosterone derivative for radiolabeling of the present invention further comprises a diagnostic and/or therapeutic radionuclide and/or a diagnostic radiotracer.

As discussed above, examples for diagnostic and/or therapeutic radionuclides are ⁶⁸Ga, ¹⁷⁷Lu, ⁶⁴Cu, ⁸⁹Zr, and ¹⁸F.

A testosterone derivative and/or a testosterone derivative for radiolabeling radiolabeled with ¹⁸F can also be a testosterone derivative and/or a testosterone derivative for radiolabeling radiolabeled with ¹⁸F-FBA (fluorobenzaldehyde).

As discussed above, an example for a diagnostic radiotracer is ¹⁸FDG (fluorodeoxyglucose).

Preferably, DOTA- and NODAGA-conjugated testosterone derivative(s) or DOTA- and NODAGA-conjugated testosterone derivative(s) for radiolabeling are radiolabeled with diagnostic and/or therapeutic radionuclides, such as ⁶⁸Ga, ¹⁷⁷Lu, ⁶⁴Cu, or ⁸⁹Zr.

Preferably, AoA-conjugated testosterone derivative(s) or AoA-conjugated testosterone derivative(s) for radiolabeling are radiolabeled with ¹⁸F or ¹⁸FDG (fluorodeoxyglucose).

In a preferred embodiment, the testosterone derivative and/or the testosterone derivative for radiolabeling of the present invention is obtained by a method of the present invention.

As outlined above, the present invention provides the use of a testosterone derivative and/or a testosterone derivative for radiolabeling of the present invention or a testosterone derivative and/or a testosterone derivative for radiolabeling obtained by a method of the present invention for radiolabeling with a diagnostic and/or therapeutic radionuclide and/or with a diagnostic radiotracer.

As outlined above, the present invention provides the testosterone derivative and/or the testosterone derivative for radiolabeling of the present invention for use in medicine.

As outlined above, the present invention provides the testosterone derivative and/or the testosterone derivative for radiolabeling of the present invention for use in a method of imaging, diagnosis and/or treatment of cancer. Such use typically comprises administering the testosterone derivative and/or the testosterone derivative for radiolabeling to a patient in need thereof.

Preferably the cancer is an androgen-receptor-positive cancer.

The androgen-receptor-positive cancer may be a cancer selected from the group comprising breast cancer (such as estrogen receptor positive and estrogen receptor negative, triple negative breast cancer, etc.), prostate cancer, lung cancer, salivary duct cancer, etc.

Preferably the cancer is an androgen-receptor-positive breast cancer, including both invasive and in situ ductal carcinomas (IDC and DCIS respectively), triple-negative breast cancer (TNBC), lobular carcinoma, BRCA-mutated tumors, and mammary Paget's disease, or prostate cancer. Even more preferably the cancer is an androgen-receptor-positive breast carcinoma or prostate carcinoma.

Preferably, the testosterone derivative and/or the testosterone derivative for radiolabeling of the present invention is used in a method of imaging, diagnosis and/or treatment of cancer in a patient. Such method typically comprises administering the testosterone derivative and/or the testosterone derivative for radiolabeling to a patient in need thereof.

The method of imaging, diagnosis and/or treatment of cancer can be used together with an ongoing therapy for the treatment of cancer.

The testosterone derivative and/or the testosterone derivative for radiolabeling of the present invention can be used in a method of imaging, diagnosis and/or treatment of cancer in combination with additional active agents or adjuvants. Such method typically comprises administering the testosterone derivative and/or the testosterone derivative for radiolabeling to a patient in need thereof.

The testosterone derivative and/or the testosterone derivative for radiolabeling of the present invention can be used for targeting of AR in combination with other active agents that target ErbB2/HER2 oncogene. Such use for targeting typically comprises administering the testosterone derivative and/or the testosterone derivative for radiolabeling to a patient in need thereof.

In one embodiment, the term “patient” relates to a human or an animal, preferably a human.

In one particular embodiment, the patient is a human that is on endocrine therapy.

For imaging and/or diagnosis, the testosterone derivative and/or the testosterone derivative for radiolabeling used is preferably radiolabeled with a diagnostic radionuclide or a diagnostic radiotracer.

Examples for diagnostic radionuclides are ⁶⁸Ga, ⁶⁴Cu, ⁸⁹Zr, ¹⁸F.

An example for a diagnostic radiotracer is ¹⁸F-FBA (fluorobenzaldehyde).

For treatment and therapy, the testosterone derivative and/or the testosterone derivative for radiolabeling used is preferably radiolabeled with a therapeutic radionuclide.

Examples for therapeutic radionuclides are ¹⁷⁷Lu and ⁶⁴Cu.

In a further aspect, the present invention also relates to the use of the testosterone derivative and/or the testosterone derivative for radiolabeling in accordance with the present invention for the manufacture of a medicament for the imaging, diagnosis and/or treatment of cancer, preferably androgen-receptor-positive cancer.

As outlined above, the present invention provides a method of imaging, diagnosis and/or treatment of cancer, preferably androgen-receptor-positive cancer; more preferably an androgen-receptor-positive breast cancer or prostate cancer; even more preferably an androgen-receptor-positive breast carcinoma or prostate carcinoma.

Said method comprises administering a diagnostic and/or therapeutic amount of a testosterone derivative and/or of a testosterone derivative for radiolabeling of the present invention.

A “therapeutic amount” or a “therapeutically effective amount” of a testosterone derivative and/or a testosterone derivative for radiolabeling of the present invention refers to the amount which has to be administered to a subject in need thereof in order to achieve a desired therapeutic result or outcome. The skilled artisan will be able to determine said therapeutically effective amount and the suitable administration regimen.

A “diagnostic or tracer amount” of a testosterone derivative and/or of a testosterone derivative for radiolabeling of the present invention refers to the amount which has to be administered to a subject in order to allow imaging and/or diagnosis in the desired high quality and high resolution. The skilled artisan will be able to determine a suitable diagnostic amount or tracer amount.

Administration of the testosterone derivative and/or the testosterone derivative for radiolabeling can be performed either once or the administration can be repeated.

The route of administration can be intravenous.

The testosterone derivative and/or the testosterone derivative for radiolabeling for use in a method of imaging, diagnosis and/or treatment of cancer may be used for the detection of a cancer. Such use typically comprises administering the testosterone derivative and/or the testosterone derivative for radiolabeling to a patient in need thereof.

The testosterone derivative and/or the testosterone derivative for radiolabeling for use in a method of imaging, diagnosis and/or treatment of cancer may be used during any stage of the cancer. These stages include the commonly used tumor stages I to IV, and all stages of the TNM-staging system, e.g. ranging from T0 to T4, from No to N3, and from M0 to M1.

The testosterone derivative and/or the testosterone derivative for radiolabeling for use in a method of imaging, diagnosis and/or treatment of cancer may be used at any stage of therapy of a subject. Such use typically comprises administering the testosterone derivative and/or the testosterone derivative for radiolabeling to a patient in need thereof.

For imaging and/or diagnosis, the testosterone derivative and/or the testosterone derivative for radiolabeling administered is preferably radiolabeled with a diagnostic radionuclide.

For treatment and therapy, the testosterone derivative and/or the testosterone derivative for radiolabeling administered is preferably radiolabeled with a therapeutic radionuclide.

As discussed above, examples for diagnostic radionuclides are ⁶⁸Ga, ¹⁷⁷Lu, ⁶⁴Cu, ⁸⁹Zr, and ¹⁸F.

As discussed above, an example for a diagnostic radiotracer is ¹⁸FDG (fluorodeoxyglucose).

As discussed above, examples for therapeutic radionuclides are ¹⁷⁷Lu and ⁶⁴Cu.

Preferably, DOTA- and NODAGA-conjugated testosterone derivative(s) or DOTA- and NODAGA-conjugated testosterone derivative(s) for radiolabeling are radiolabeled with diagnostic and/or therapeutic radionuclides, such as ⁶⁸Ga, ¹⁷⁷Lu, ⁶⁴Cu, or ⁸⁹Zr.

Preferably, AoA-conjugated testosterone derivative(s) or AoA-conjugated testosterone derivative(s) for radiolabeling are radiolabeled with ¹⁸F or ¹⁸FDG (fluorodeoxyglucose).

In this invention, the present inventors present a novel acetic acid testosterone of the general formula 1 and means of producing said acetic acid testosterone. Furthermore, the inventors present the use of this novel acetic acid testosterone in the novel solid-phase synthesis of testosterone derivative(s) and/or the testosterone derivative(s) for radiolabeling, wherein the acetic acid testosterone is linked or conjugated with one of three different bifunctional groups, namely DOTA (1,4,7,10-tetraazacyclododecane-1,4,7,10-tetraacetic acid), NODAGA (1,4,7-triazacyclononane-1-glutaric acid-4,7-diacetic acid), and AoA (aminooxyacetic acid). This linking or conjugating can also involve the use of a PEG (Fmoc-8-amino-3,6-dioxaoctanoic acid, 9 atoms) as a spacer in the testosterone derivative molecule(s) to increase the overall hydrophilicity, enhance its metabolic stability by possible reduction in enzymatic proteolysis, and increase the in vivo half-life of the testosterone derivative(s).

The DOTA- and NODAGA-conjugated testosterone derivatives and/or testosterone derivatives for radiolabeling and/or the DOTA-PEG- and NODAGA-PEG-conjugated testosterone derivatives and/or testosterone derivatives for radiolabeling will allow the radiolabeling with a variety of medically useful diagnostic and therapeutic radionuclides (i.e., ⁶⁸Ga, ¹⁷⁷Lu, ⁶⁴Cu, ⁸⁹Zr, etc.) both for diagnosis and therapy.

Also, AoA-conjugated testosterone derivatives and/or testosterone derivatives for radiolabeling or AoA-PEG-conjugated testosterone derivatives and/or testosterone derivatives for radiolabeling can be radiolabeled with ¹⁸F and ¹⁸FDG (fluorodeoxyglucose) via oxime functionality.

These new testosterone derivatives and/or testosterone derivatives for radiolabeling hold the ability to be new theranostic agents for both imaging and therapeutic applications for androgen-receptor-expressing primary and metastatic cancers.

The development of new theranostic-based testosterone radiopharmaceuticals will be beneficial for the efficient management of androgen-receptor-expressing cancers, such as androgen-receptor-positive breast cancer or androgen-receptor-positive prostate cancer.

The invention discloses a novel acetic acid testosterone of the general formula 1 and means for producing said acetic acid testosterone. Furthermore, the invention discloses an easy and effective method of preparing (a) testosterone derivative(s) and/or a testosterone derivative(s) for radiolabeling. The solid-phase synthesis technique holds the distinct advantage of preparing testosterone derivatives and/or testosterone derivatives for radiolabeling as it can be synthesized efficiently in fewer synthetic steps with few purification steps. It has added advantages of solubility in aqueous solution, reagents used are less expensive, less reagents are required, synthesis is rapid, and provides testosterone derivatives in good purity. The preparation method is novel as claimed for the convenient and economic preparation of DOTA-testosterone or NODAGA-testosterone or AoA-testosterone or DOTA-PEG-testosterone or NODAGA-PEG-testosterone or AoA-PEG-testosterone.

The method disclosed by present invention has the following beneficial effects:
i) the reagents used are less expensive,
ii) the reaction times are short,
iii) the required reagents of reaction consumes little,
iv) fewer preparation steps,
v) fewer purification steps,
vi) flexible synthesis approach,
vii) different bifunctional ligands can be conjugated,
viii) allows radiolabeling with different radionuclides,
ix) better solubility in the aqueous medium,
x) high purity,
xi) can be used as a small-scale (milligrams) or large-scale (grams) preparation, and
xii) suitability for industrialized production.

### Further Description of Preferred Embodiments

Testosterone is a male sex hormone that binds to androgen receptors (Ars) which are overexpressed in cancers, such as prostate and breast carcinoma. Radiolabeled compounds specifically designed to image tumors, such as breast and prostate tumors based on the androgen content (i.e., testosterone) may serve as a useful clinical tool in staging these AR-positive caner and monitoring the course of hormonal therapy. This invention relates to a novel acetic acid testosterone. This invention further relates to a novel and convenient preparation method for testosterone derivatives and/or testosterone derivatives for radiolabeling using said acetic acid testosterone. Preferably, the novel preparation method for testosterone derivatives and/or testosterone derivatives for radiolabeling comprises the following synthetic steps:
(1) preparing an acetic acid testosterone of the general formula 1;
(2) either
   (a) coupling the acetic acid testosterone obtained in step (1) to a solid-phase synthesis resin, preferably Wang resin, wherein said resin, preferably Wang resin, is suitable for Fmoc (9-fluorenylmethyl-oxycarbonyl)-solid-phase synthesis and carries a Fmoc-lysine with a protecting group at the lysine side chain;
   or
   (a′) first coupling Fmoc-NH-PEG-COOH (9 atoms; 4 equivalent) to a solid-phase synthesis resin, preferably Wang resin, wherein said resin, preferably Wang resin, is suitable for Fmoc (9-fluorenylmethyl-oxycarbonyl)-solid-phase synthesis and carries Fmoc-lysine with a protecting group at the lysine side chain to obtain a Fmoc-NH-PEG-resin, preferably a Fmoc-NH-PEG-Wang resin, and second, coupling the acetic acid testosterone obtained in step (1) to the Fmoc-NH-PEG-resin, preferably Fmoc-NH-PEG-Wang resin;
(3) removing the protective group at the lysine side chain;
(4) coupling a bifunctional group for radiolabeling to the lysine side chain; and
(5) removing the testosterone derivative and/or the testosterone derivative for radiolabeling from the resin.

Preferably the bifunctional groups in step (4) are DOTA or NODAGA or AoA to prepare DOTA/NODAGA/AoA-testosterone derivatives, or DOTA-PEG/NODAGA-PEG/AoA-PEG-testosterone derivatives, or DOTA/NODAGA/AoA-testosterone derivatives for radiolabeling, or DOTA-PEG/NODAGA-PEG/AoA-PEG-testosterone derivatives for radiolabeling with both diagnostic and therapeutic radionuclides, thus developing an image- and treat-based theranostic approach. When the testosterone derivative or the testosterone derivative for radiolabeling is prepared with this inventive method, the reagents used for the preparation are less expensive, the reaction time is short, the consumption of the reagents required by the reaction is small, and fewer preparation and purification steps give high-yield preparation, and the novel testosterone derivative preparing method is suitable for industrial production. This invention represents the design of radiolabeled testosterone-related compounds for the targeting of breast and other AR-positive cancers.

### Step (1) the Preparation of Acetic Acid Testosterone (3):

The preparation of acetic acid testosterone is presented in FIG. 1.

Testosterone (1) (3.0 mmol, 866 mg) is dissolved in 10 mL of DMF (N,N-dimethylformamide). To this potassium carbonate (K₂CO₃, 500 mg, 1.2 equivalent) is added and the mixture is stirred for 5 minutes at room temperature. Bromoacetic acid (2) (420 mg, 1.0 equivalent) is then added followed by the addition of potassium iodide catalyst (KI, 50 mg, 0.1 equivalent). The reaction mixture is heated at 80-90° C. for 4 hours and then stirred at ambient temperature for 18 hours to ensure quantitative coupling. The solvents are removed on a rotary evaporator, and the residue is diluted with ethyl acetate (25 mL) and transferred to a separatory funnel. The organic layer is washed with 2×20 mL of 10% (w/v) sodium hydroxide solution (to remove unreacted testosterone-OH) in the solvent extraction method. The organic layer is then separated and dried with anhydrous sodium sulfate for 30 minutes. The solvent is filtered off using a filter paper and the filtrate is evaporated under reduced pressure to provide the alkylated testosterone product. The acetic acid testosterone (3) (=general formula 1) is dried in a desiccator for 18 hours to yield the desired compound as a solid.

The solid-phase synthesis of the testosterone derivative or the testosterone derivative for radiolabeling is started by utilizing the commercially available Fmoc-Lys(ivDde)-Wang resin using the peptide synthesis glass reaction vessel (Peptides International, Louisville, USA). For solid-phase synthesis, standard Fmoc (9-fluorenylmethyl-oxycarbonyl) chemistry was employed on a 0.2 mmol scale.

### Removal of the Fmoc Protecting Group of the Fmoc-Lys(ivDde)-Wang Resin

Removal of the Fmoc protecting group of the Fmoc-Lys(ivDde)-Wang resin is presented in FIG. 2A. The Lys(ivDde)-Wang resin is soaked in 5 mL DMF for 30 minutes and in 5 mL DCM (dichloromethane) for another 30 minutes. The solvents are drained and the Fmoc protecting group of the lysine is removed by treating the resin with 3 mL of 20% (v/v) piperidine solution in DMF (1×5 minutes, 1×10 minutes) followed by standard washing with DMF (5×3 mL), DCM (3×3 mL) and DMF (2×3 mL).

### Step (2) (a)

### Coupling of Acetic Acid Testosterone to NH₂-Lys(ivDde)-Wang Resin

To attach testosterone to the Wang resin, the acetic acid testosterone (3) (=general formula 1) (4-times molar excess as compared with the resin) dissolved in 5 mL DMF is first preactivated for 5 minutes at its α-carboxylic function by the use of an activating reagent, HATU (hexafluorophosphate azabenzotriazole tetramethyl uronium) and oxyma pure (ethyl cyanohydroxyiminoacetate) in the presence of a base DIEA (diisopropylethylamine) and then allowed to react for 3 hours at the ambient temperature with the resin-bound amino group of lysine to form an amide bond resulting in the compound 3a (FIG. 2B). The completion of the coupling is confirmed by the Kaiser ninhydrin test, as known to the skilled artisan.

Step (2) (a′)
Coupling of PEG with NH₂-Lys(ivDde)-Wang Resin

Fmoc-NH-PEG-COOH (9 atoms; 4 equivalent) dissolved in 5 mL DMF is first preactivated for 5 minutes at its α-carboxylic function by the use of an activating reagent, HATU, and oxyma pure in the presence of a base DIEA and then allowed to react for 2 hours at the ambient temperature with the resin-bound amino group of lysine to form an amide bond. This results in the formation of a Fmoc-NH-PEG-Lys(ivDde)-Wang resin. The completion of the coupling is confirmed by the Kaiser ninhydrin test. The resin is washed with DMF (3×3 mL), and DCM (3×3 mL).

### Removal of Fmoc Protecting Group of PEG

Fmoc protecting group of the PEG is removed by treating the resin with 3 mL of 20% (v/v) piperidine solution in DMF (1×5 minutes, 1×10 minutes) followed by standard washing with DMF (5×3 mL), DCM (3×3 mL) and DMF (2×3 mL). This results in the formation of a NH2-PEG-Lys(ivDde)-Wang resin.

### Coupling of Acetic Acid Testosterone to NH2-PEG-Lys(ivDde)-Wang Resin

To attach testosterone to the NH2-PEG-Lys(ivDde)-Wang resin, the acetic acid testosterone (3) (4-times molar excess as compared with the resin) in 5 mL DMF was first preactivated for 5 minutes at its α-carboxylic function by the use of an activating reagent, HATU and oxyma pure in the presence of a base DIEA and then allowed to react for 3 hours at the ambient temperature with the resin-bound amino group of lysine to form an amide bond. The completion of the coupling is confirmed by the Kaiser ninhydrin test.

Step (3) Removal of the ivDde Protecting Group

At this point, the ivDde (1-(4,4-dimethyl-2,6-dioxocyclohex-1-ylidene)-3-methylbutyl) protecting group of the lysine is deprotected using a 3% (v/v) hydrazine monohydrate in DMF (4×3 mL, 5 minutes each) followed by standard washings with DMF (3×3 mL) and DCM (3×3 mL) to allow conjugation with a bifunctional group, e.g. for the radiolabeling purpose, (i.e., DOTA or NODAGA or AoA), as described below. This is exemplified by compound 3b which represents the resulting compound after removal of the ivDde protecting group of compound 3a of step (2) (a) (FIG. 2C). Compound 3b can be used for production of DOTA-Testosterone (4), NODAGA-Testosterone (5), AoA-Testosterone (6) (see below).

### Step (4) Coupling of a Bifunctional Group for Radiolabeling to the Lysine Side Chain

### Coupling of DOTA to Acetic Acid Testosterone-Lysine-Wang Resin

For the preparation of DOTA (1,4,7,10-tetraazacyclododecane-1,4,7,10-tetraacetic acid) coupled with testosterone, available commercially DOTA-tris-(t-Bu ester) (1,4,7,10-tetraazacyclododecane-1,4,7-tris-tert-butyl acetate-10-acetic acid) is first preactivated with HATU (2.5 equivalent) and DIEA (5 equivalent) in 5 mL DMF for 15 minutes before the conjugation with the resin. The coupling mixture is allowed to react for 5 hours at ambient temperature and the completion of the reaction is confirmed by the negative Kaiser test. The resin is washed with DMF (3×3 mL), and DCM (3×3 mL). DOTA-testosterone-lysine-Wang resin is obtained as a solid product.

### Coupling of NODAGA to Acetic Acid Testosterone-Lysine-Wang Resin

For the preparation of NODAGA (1,4,7-triazacyclononane-1,4,7-triacetic acid) conjugation with testosterone, commercially available NODAGA-tris-(t-Bu ester) 1,4,7-triazacyclononane-1,4-bis-tert-butyl acetate-7-acetic acid (2.5 equivalent) is first preactivated with HATU (2.5 equivalent) and DIEA (5 equivalent) in 5 mL DMF for 15 minutes before the conjugation with the resin. The coupling mixture is allowed to react for 5 hours at ambient temperature and the completion of the reaction is confirmed by the negative Kaiser test. The resin is washed with DMF (3×3 mL), and DCM (3×3 mL). NODAGA-testosterone-lysine-Wang resin is obtained as a solid product.

### Coupling of AoA to Acetic Acid Testosterone-Lysine-Wang Resin

For the coupling to tert-butyloxycarbonyl-aminooxy acetic acid (Boc-AoA), Boc-AoA is first pre-activated with HATU (4 equivalent) and DIEA (8 equivalent) in 5 mL DMF for 5 minutes before the conjugation with the resin. The coupling reaction is completed after 2 hours, as determined by the negative Kaiser test. The resin is washed with DMF (3×3 mL), DCM (3×3 mL), and ether (2×3 mL) and dried for 18 hours in a desiccator. AoA-testosterone-lysine-Wang resin is obtained as a solid product.

### Coupling of DOTA to Acetic Acid Testosterone-PEG-Lysine-Wang Resin

It has been shown that small modifications in the bioactive molecular targeting vectors can influence their biodistribution pattern and tumor targeting properties. Polyethylene glycol (PEG) has been widely used for modification of therapeutic peptides. In the present application, the inventors have also introduced a PEG (Fmoc-8-amino-3,6-dioxaoctanoic acid, 9 atoms) as a spacer in the testosterone molecule to increase the overall hydrophilicity, enhance its metabolic stability by possible reduction in enzymatic proteolysis, and increase the in vivo half-life of testosterone.

For the preparation of DOTA-PEG-linked testosterone, the commercially available DOTA-tris-(t-Bu ester) is first preactivated with HATU (2.5 equivalent) and DIEA (5 equivalent) in 5 mL DMF for 15 minutes before the conjugation with the resin. The coupling mixture is allowed to react for 5 hours at the ambient temperature. The completion of the reaction is confirmed by the negative Kaiser test. The resin is washed with DMF (3×3 mL), and DCM (3×3 mL). DOTA-PEG-testosterone-lysine-Wang resin is obtained as a solid product.

### Coupling of NODAGA or AoA to Acetic Acid Testosterone-PEG-Lysine-Wang Resin

The preparation of NODAGA-PEG-testosterone-lysine-Wang resin or AoA-PEG-testosterone-lysine-Wang resin is conducted analogously to the respective reactions described for the coupling of NODAGA or AoA to acid testosterone-lysine-Wang resin.

Step (5) Removal of the testosterone derivative(s) from the resin and side-chain deprotection: Finally, the testosterone derivative(s), such as DOTA-testosterone, NODAGA-testosterone, AoA-testosterone, DOTA-PEG-testosterone, NODAGA-PEG-testosterone, and AoA-PEG-testosterone are cleaved from the resin along with other side-chain protecting groups by reacting the resin with a cleavage cocktail (˜5 mL) of trifluoroacetic acid (TFA)/triisopropylsilane/water (95%:2.5%:2.5%, v/v) for 4 hours at ambient temperature. The resin is removed by filtration and TFA is evaporated using a rotary evaporator. To the residue, cold diethyl ether is added to precipitate the desired product which is then transferred to a 15 mL centrifuge vial and centrifuged thrice at 7200 RPM to form a pellet of the target product. The ether is decanted and the DOTA/NODAGA/AOA-testosterone or the DOTA-PEG/NODAGA-PEG/AoA-PEG-testosterone products are allowed to dry overnight in a desiccator to yield the desired testosterone analogs as solid.

FIGS. 3-6 show preferred structures of the resulting DOTA-Testosterone (4) (FIG. 3), NODAGA-Testosterone (5) (FIG. 4), AoA-Testosterone (6) (FIG. 5), and DOTA-PEG-Testosterone (7) (FIG. 6), respectively. The purity of these testosterone compounds can be checked by reversed-phase high-performance liquid chromatography and their structural identities can be checked by mass spectrometry analysis.

### BRIEF DESCRIPTION OF THE FIGURES

The present invention is now further described by reference to the following figures.

All methods mentioned in the figure descriptions below were carried out as described in detail in the examples.

FIG. 1: A preferred chemical synthesis of acetic acid testosterone (3).

FIGS. 2A-2C: A preferred preparation of DOTA/NODAGA/AoA-testosterone by solid-phase synthesis.

FIG. 3: Preferred structure of DOTA-Testosterone (4).

FIG. 4: Preferred structure of NODAGA-Testosterone (5).

FIG. 5: Preferred structure of AoA-Testosterone (6).

FIG. 6: Preferred structure of DOTA-PEG-Testosterone (7).

FIG. 7: Mass spectrometry of Acetic acid Testosterone (3).

LC-MS: m/z calculated for C₂₁H₃₀O₄ 346.4; found, 346.8 [M+H]⁺.

FIG. 8: Mass spectrometry of DOTA-Testosterone (4).

LC-MS: m/z calculated for C₄₃H₆₉N₆O₁₂ 861.4; found, 861.5 [M+H]⁺.

FIG. 9: Mass spectrometry of NODAGA-Testosterone (5).

LC-MS: m/z calculated for C₄₂H₆₅N₅O₁₂ 831.5; found, 832.3.

FIG. 10: Mass spectrometry of AoA-Testosterone (6).

LC-MS: m/z calculated for C₂₉H₄₆N₃O₇ 548.7; found, 549.8 [M+H]⁺.

FIG. 11: Mass spectrometry of DOTA-PEG-Testosterone (7).

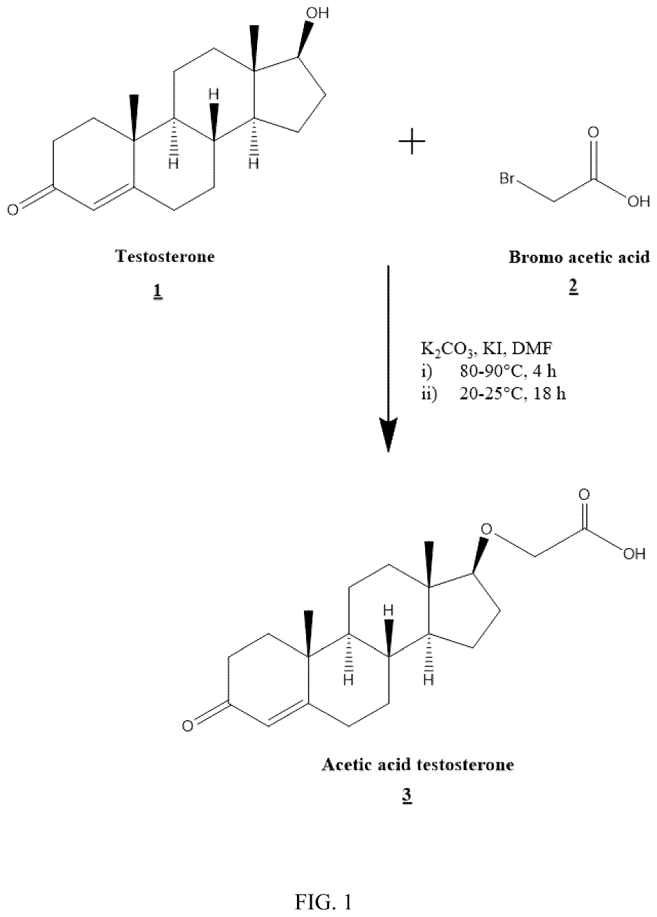

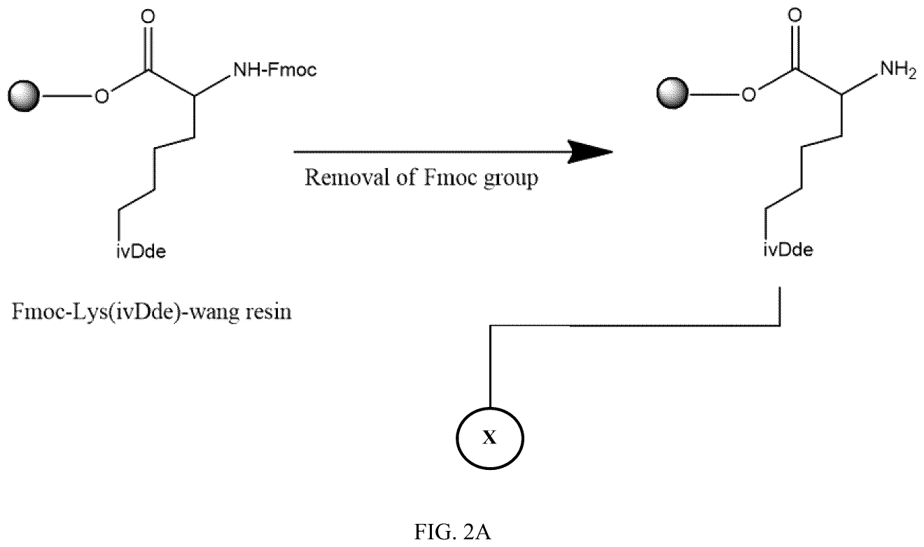

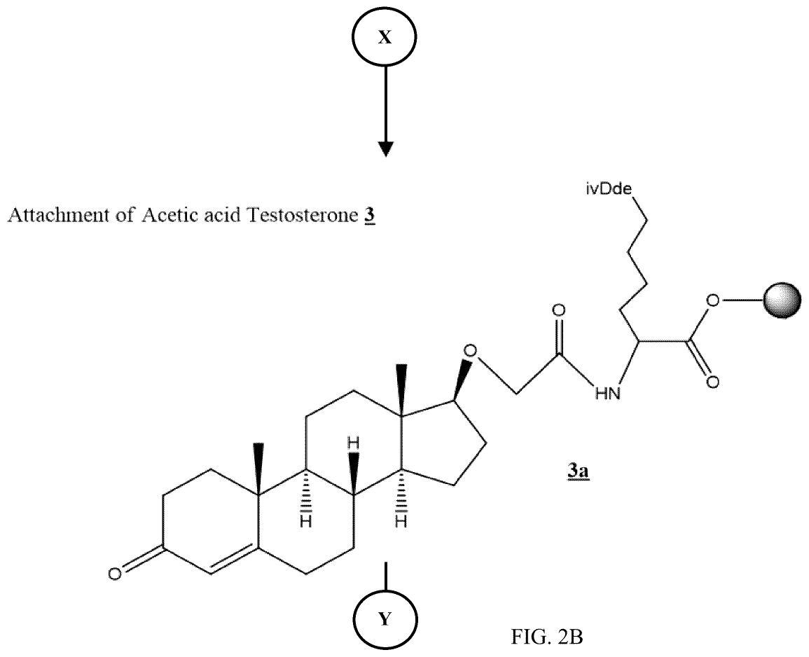

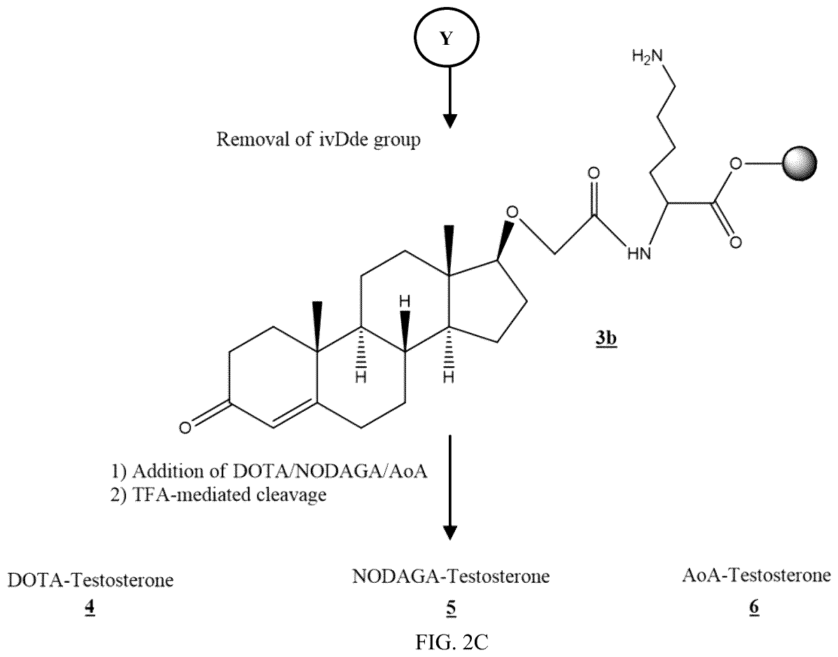

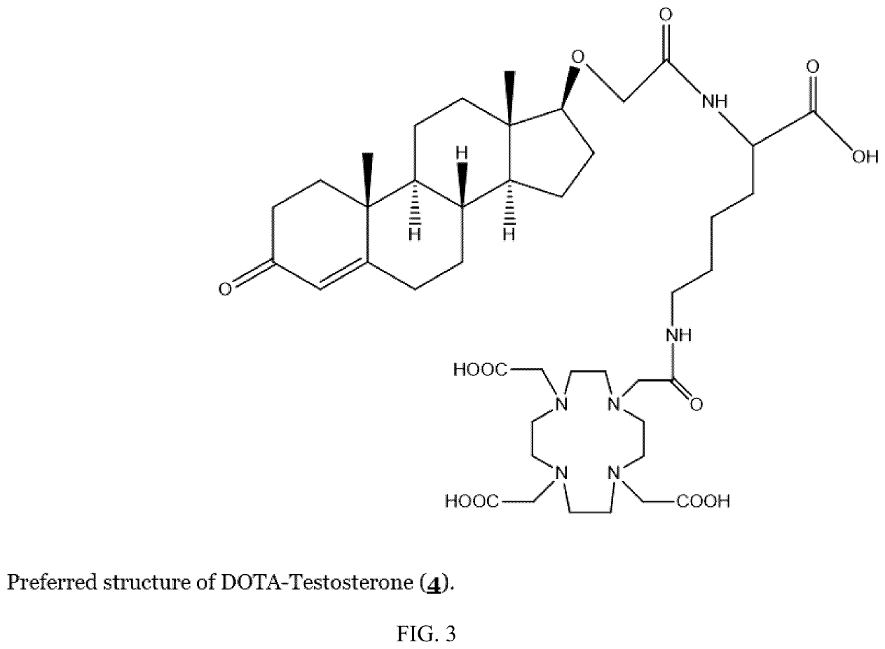

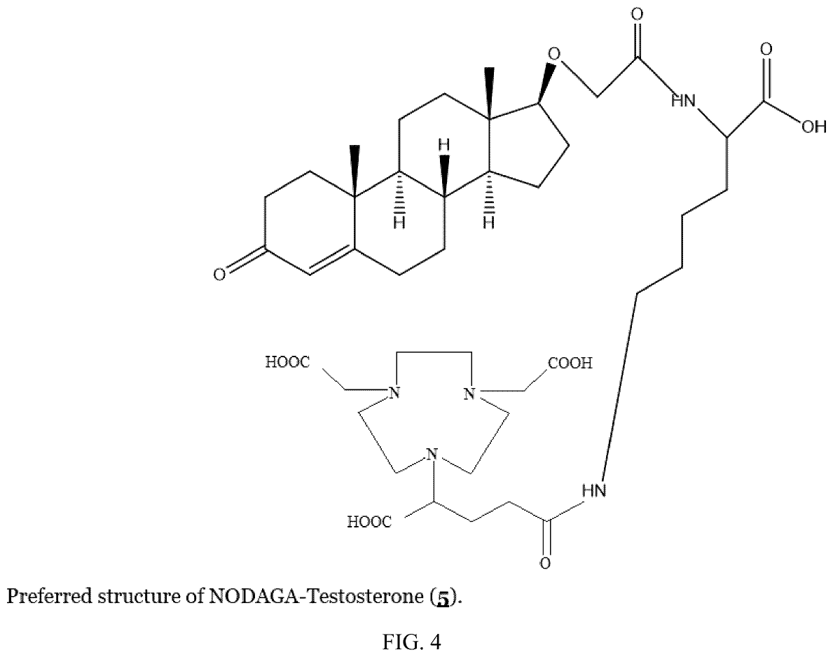

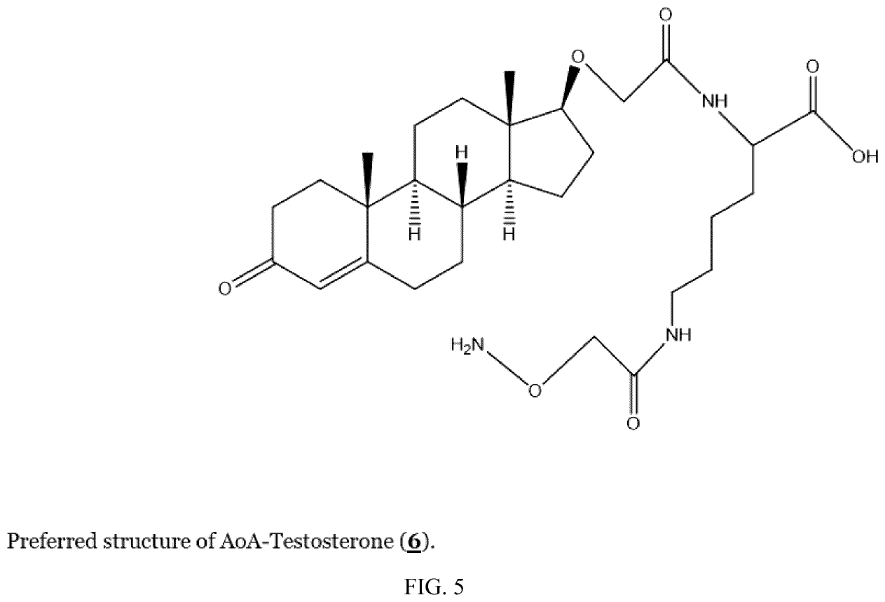

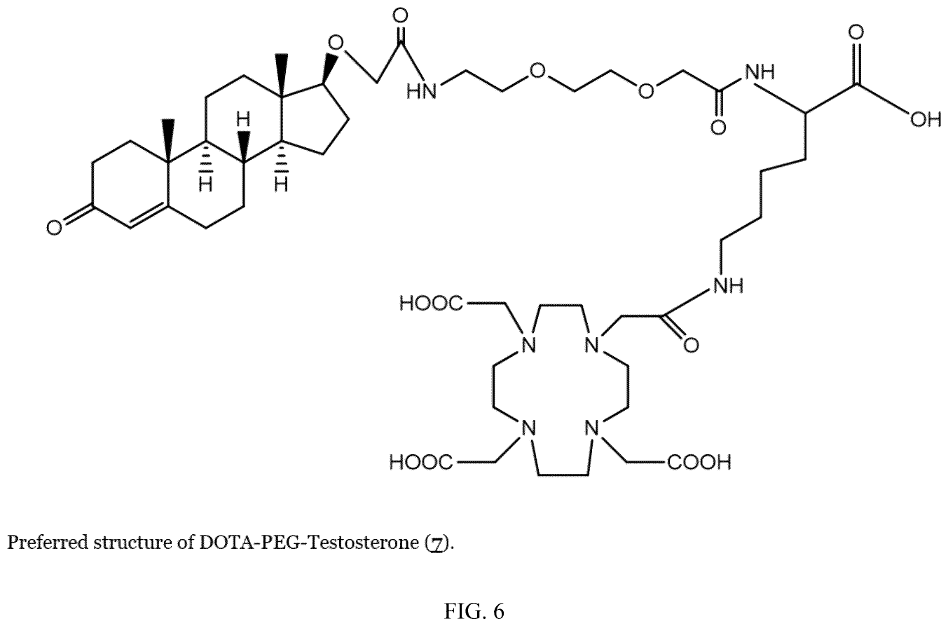

LC-MS: m/z calculated for C₅₂H₇₆N₂O₁₃ 1006.5; found, 1006.6 [M+H]⁺.

In the following, reference is made to the examples, which are given to illustrate, not to limit the present invention.

### EXAMPLES

### Example 1

Acetic acid testosterone was prepared according to section “Step (1) The preparation of acetic acid testosterone (3)”.

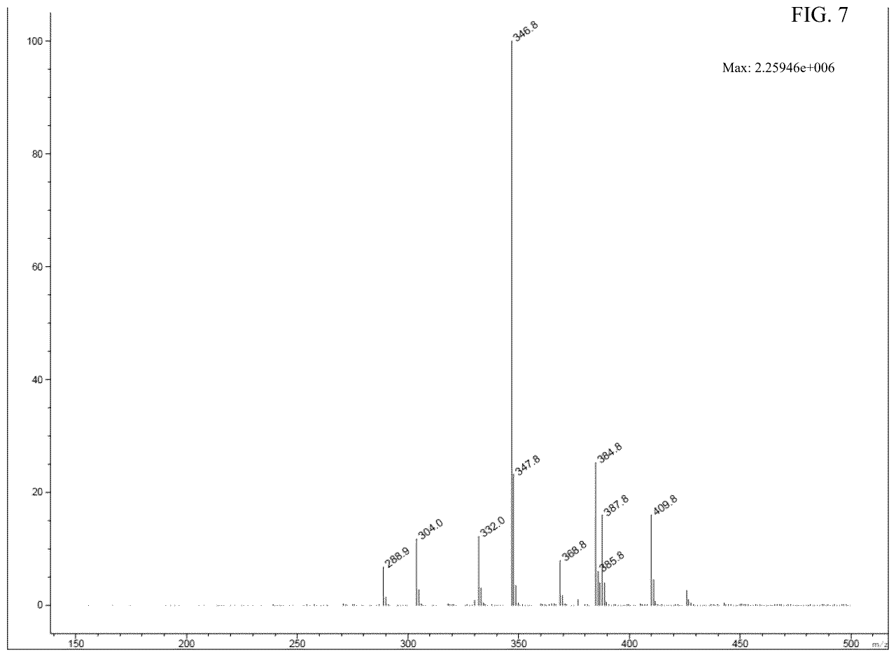

The structural identity of the acetic acid testosterone (3) was confirmed by liquid chromatography-mass spectrometry (LC-MS) analysis (FIG. 7).

Yield=60%; C₂₁H₃₀O₄=MW: 346.4.

LC-MS: m/z calculated for C₂₁H₃₀O₄ 346.4; found, 346.8 [M+H]⁺.

### Example 2

DOTA-testosterone derivatives, NODAGA-testosterone derivatives, AoA-testosterone derivatives, or the DOTA-PEG-testosterone derivative were prepared according to the present invention.

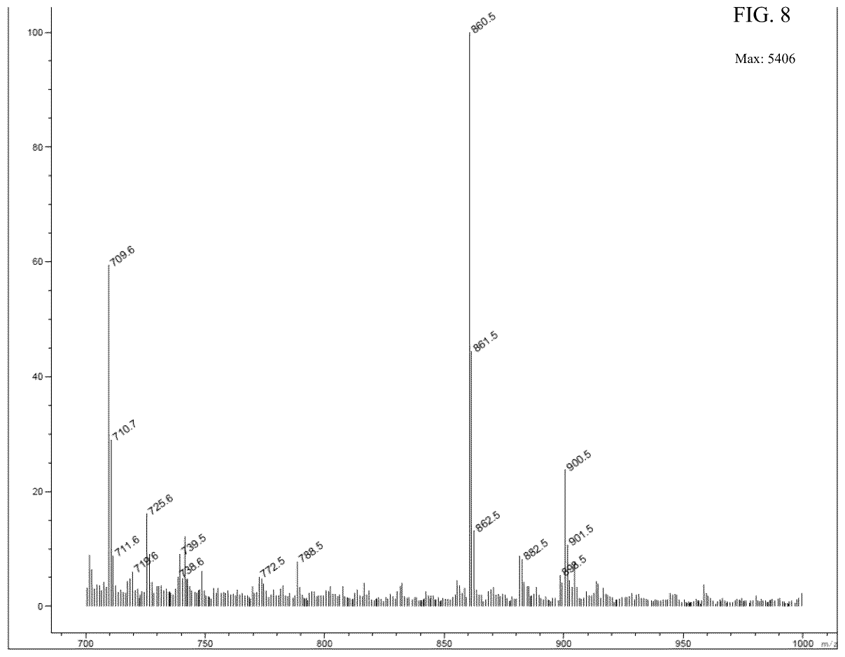

The purity of the newly synthesized DOTA/NODAGA/AoA-testosterone derivatives and the DOTA-PEG-testosterone derivative was checked by reversed-phase high-performance liquid chromatography (RP-HPLC) and their structures were confirmed by mass spectrometry analyses performed on Agilent 6125 single quadrupole liquid chromatography/mass spectrometry system (LC/MS) (Agilent Technologies, Santa Clara, CA, USA) using an eluent of 0.1% formic acid/29.95% water/69.95% acetonitrile at a flow rate of 0.3 mL/min. It was found that the experimentally determined molecular weights correlated well with the theoretically calculated values (FIGS. 8-11).

For DOTA-Testosterone (4) (FIG. 8):

Yield=40%; C₄₃H₆₉N₆O₁₂=MW: 861.4

LC-MS: m/z calculated for C₄₃H₆₉N₆O₁₂ 861.4; found, 861.5 [M+H]⁺.

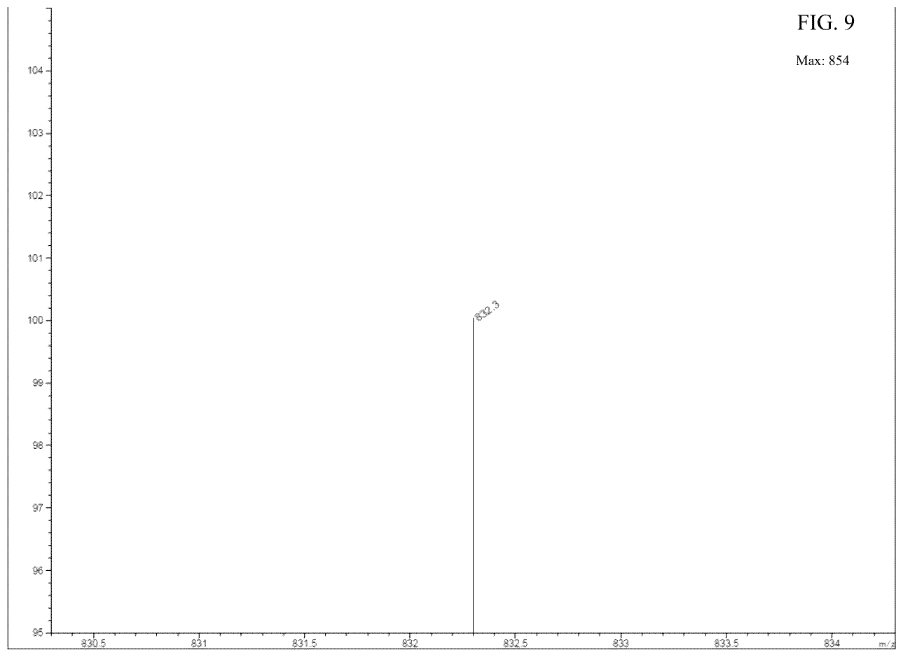

For NODAGA-Testosterone (5) (FIG. 9):

Yield=37%; C₄₂H₆₅N₅O₁₂=MW: 831.5

LC-MS: m/z calculated for C₄₂H₆₅N₅O₁₂ 831.5; found, 832.3 [M+H]⁺.

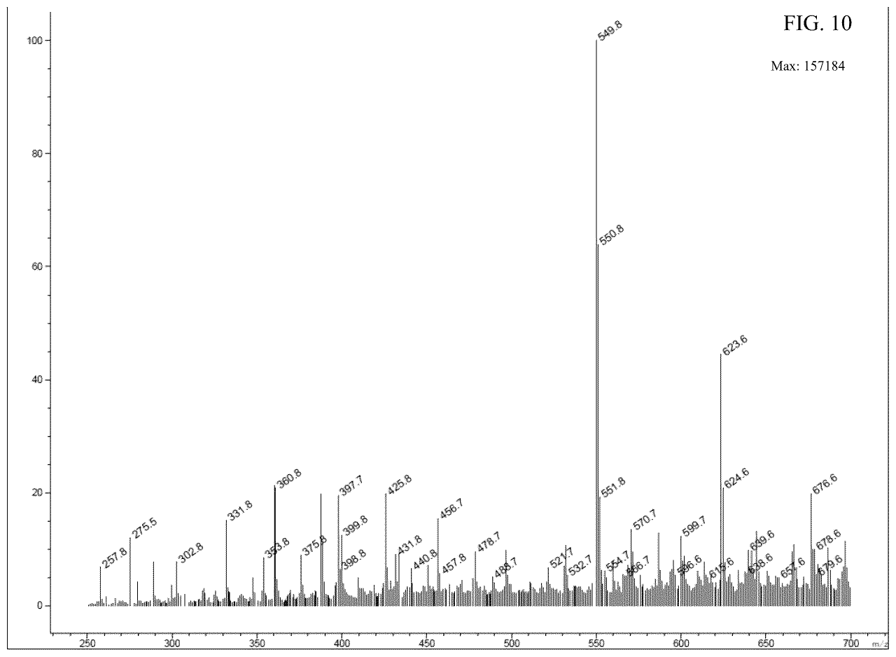

For AoA-Testosterone (6) (FIG. 10):

Yield=42%; C₂₉H₄₆N₃O₇=MW: 548.7

LC-MS: m/z calculated for C₂₉H₄₆N₃O₇ 548.7; found, 549.8 [M+H]⁺.

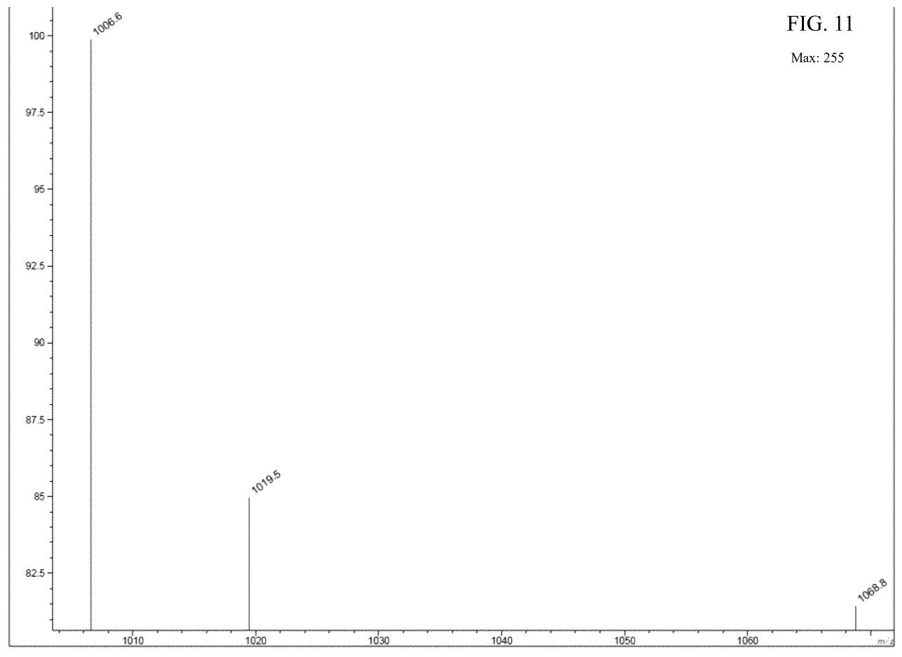

For DOTA-PEG-Testosterone (7) (FIG. 11):

Yield=40%; C₅₂H₇₆N₂O₁₃=MW: 1006.5

LC-MS: m/z calculated for C₅₂H₇₆N₂O₁₃ 1006.5; found, 1006.6 [M+H]⁺.

In summary, the present invention provides a novel acetic acid testosterone and means for producing said acetic acid testosterone. This acetic acid testosterone can be used for adopting a novel testosterone derivative preparation method provided by this invention to prepare testosterone derivatives by solid-phase synthesis. The reagents that this preparation uses are cheap, the reaction time is short, and the required reagents of the reaction consume little, fewer preparation and purification steps, flexible synthetic approach, suitable for industrialized production. This invention is the first step toward the development of testosterone derivatives and the scope of the invention can be extended for its application in the imaging and treatment of androgen-receptor-positive breast and prostate carcinomas using the newly synthesized testosterone derivatives.

1. An acetic acid testosterone of the general formula 1

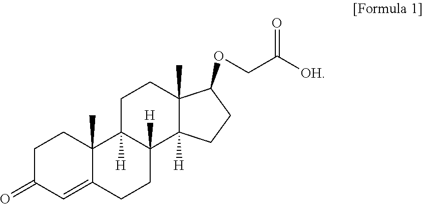

2. A method for preparing the acetic acid testosterone according to claim 1, wherein the acetic acid testosterone is prepared by reacting testosterone and bromo acetic acid; and wherein the testosterone and bromo acetic acid are reacted in the presence of potassium carbonate (K₂CO₃) and potassium iodide (KI).

3. The method according to claim 2, wherein the acetic acid testosterone is prepared by reacting testosterone and bromo acetic acid, wherein the reaction is first carried out at a temperature in a range from about 60° C. to about 100° C., for a duration in a range from about 1 h to about 10 h,
and then the reaction is carried out at a temperature in a range from about 18° C. to about 30° C., for a duration in a range from about 12 h to about 24 h.

4. A testosterone derivative wherein the testosterone derivative has the general formula 2 or the general formula 3 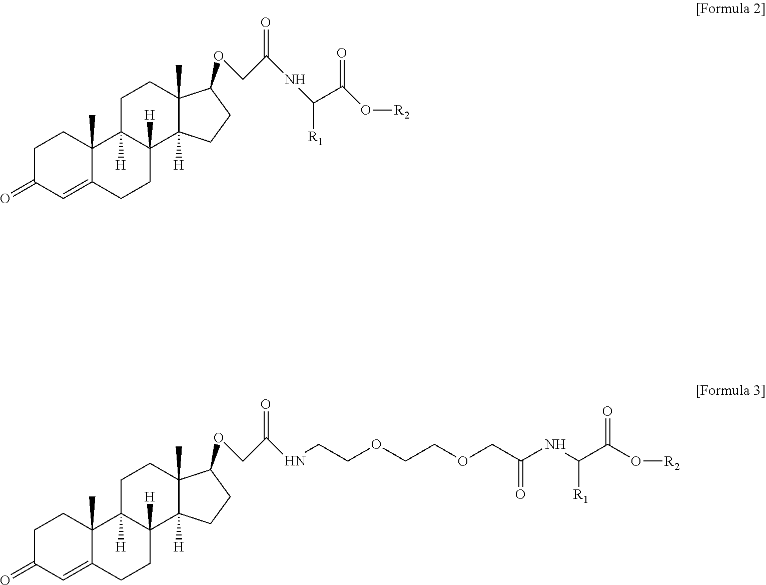
wherein R₁ is selected from a lysine side chain, a modified lysine side chain, and a bifunctional group for radiolabeling, coupled to a lysine side chain; and
wherein R₂ is selected from a resin for solid-phase synthesis and H.

5. The testosterone derivative according to claim 4, wherein the bifunctional group for radiolabeling is selected from DOTA (1,4,7,10-tetraazacyclododecane-1,4,7,10-tetraacetic acid), NODAGA (1,4,7-triazacyclononane-1-glutaric acid-4,7-diacetic acid), and AoA (aminooxyacetic acid) and/or wherein the modified lysine side chain is a lysine side chain having a protective group.

6. The testosterone derivative according to claim 4, wherein the testosterone derivative is radiolabeled with diagnostic and/or therapeutic radionuclides(s), and/or diagnostic radiotracer(s).

7. The testosterone derivative according to claim 4, wherein the testosterone derivative is selected from:
DOTA-testosterone,
NODAGA-testosterone,
AoA-testosterone,
DOTA-PEG-testosterone,
NODAGA-PEG-testosterone, and
AoA-PEG-testosterone;
wherein, preferably, the testosterone derivative and/or the testosterone derivative for radiolabeling is selected from:
DOTA-testosterone,
NODAGA-testosterone,
AoA-testosterone, and
DOTA-PEG-testosterone;
wherein, optionally, the testosterone derivative further comprises diagnostic and/or therapeutic radionuclides(s), and/or diagnostic radiotracer(s).

8. A method of preparing a testosterone derivative, wherein the method comprises the steps of
(1) providing an acetic acid testosterone according to claim 1;
(2) either
(a) coupling the acetic acid testosterone from step (1) to a solid-phase synthesis resin, wherein said resin is suitable for Fmoc (9-fluorenylmethyl-oxycarbonyl)-solid-phase synthesis and carries a Fmoc-lysine with a protecting group at the lysine side chain;
or
(a′) first coupling Fmoc-NH-PEG-COOH (9 atoms; 4 equivalent) to a solid-phase synthesis resin, wherein said resin is suitable for Fmoc (9-fluorenylmethyl-oxycarbonyl)-solid-phase synthesis and carries Fmoc-lysine with a protecting group at the lysine side chain to obtain a Fmoc-NH-PEG-resin and second, coupling the acetic acid testosterone from step (1) to the Fmoc-NH-PEG-resin;
(3) removing the protective group at the lysine side chain;
(4) coupling a bifunctional group for radiolabeling to the lysine side chain; and
(5) removing the testosterone derivative from the resin.

9. The method according to claim 8, wherein the protective group at the lysine side chain of the Fmoc-lysine is ivDde (1-(4,4-dimethyl-2,6-dioxocyclohex-1-ylidene)-3-methylbutyl) and the resin is Fmoc-Lys(ivDde)-Wang resin, or wherein the protective group at the lysine side chain of the Fmoc-lysine is Alloc (Allyloxycarbonyl) and the resin is Fmoc-Lys(Alloc)-Wang resin.

10. The method according to claim 8, wherein, prior to the coupling of step (2), a Fmoc group of a Fmoc-Lys(ivDde)-Wang resin is removed;
wherein prior to the coupling of step (2) the acetic acid testosterone and/or Fmoc-NH-PEG-COOH is preactivated at a α-carboxylic function by the use of an activating reagent; and
wherein, in case of step (2) (a′), the Fmoc group of the Fmoc-NH-PEG-resin is removed prior to coupling of the preactivated acetic acid-testosterone to said Fmoc-NH-PEG-resin.

11. The method according to claim 8, wherein the bifunctional group for radiolabeling is selected from DOTA (1,4,7,10-tetraazacyclododecane-1,4,7,10-tetraacetic acid), NODAGA (1,4,7-triazacyclononane-1-glutaric acid-4,7-diacetic acid), and AoA (aminooxyacetic acid).

12. The method according to claim 8, wherein in step (4), for coupling the bifunctional group for radiolabeling to the lysine side chain, DOTA-tris-(t-Bu ester), NODAGA-tris-(t-Bu ester), or Boc-AoA is used.

13. The method of claim 8, wherein at least one of steps (2) to (5) is carried out at a temperature in a range from about 18° C. to about 30° C.

14. The method according to claim 8, further comprising a step of
(6) radiolabeling the testosterone derivative obtained in step (5) with diagnostic and/or therapeutic radionuclide(s),
and/or diagnostic radiotracer(s).

15. A pharmaceutical composition comprising the testosterone derivative of claim 4, and a pharmaceutically acceptable carrier.

16. A method of imaging, diagnosing, and/or treating cancer, wherein said method comprises administering, to a patient in need thereof, the testosterone derivative according to claim 4.

17. The method according to claim 16, wherein the cancer is an androgen-receptor-positive cancer.

18. The method according to claim 17, wherein the androgen-receptor-positive cancer is an androgen-receptor-positive breast cancer or prostate cancer.

19. The testosterone derivative according to claim 6, wherein the testosterone is radiolabeled with a radionuclide selected from ⁶⁸Ga, ¹⁷⁷Lu, ⁶⁴Cu, ⁸⁹Zr, and ¹⁸F, and/or the testosterone derivative is radiolabeled with ¹⁸FDG (fluorodeoxyglucose) as a radiotracer.

20. The method according to claim 10, wherein said activating reagent is HATU (hexafluorophosphate azabenzotriazole tetramethyl uronium) and oxyma pure (ethyl cyanohydroxyiminoacetate) in the presence of a base DIEA (diisopropylethylamine);
or in case of step (2) (a′), the Fmoc group of the Fmoc-NH-PEG-resin is removed prior to coupling of the preactivated acetic acid-testosterone to said Fmoc-NH-PEG-resin by treating the Fmoc-NH-PEG-resin with a piperidine solution.
